(19) United States
(12) Patent Application Publication
(10) Pub. No.: US 2007/0088194 A1
(43) Pub. Date: Apr. 19, 2007

(54) Bolus, method and system for monitoring health condition of ruminant animals

(76) Inventors: Eliav Tahar, Tel Aviv (IL); Mike Nathanson, Haifa (IL)

Correspondence Address: Gary M. Nath;NATH & ASSOCIATES PLLC, 112 S. West Street, Alexandria, VA, 22314, US

(21) Appl. No.: 11/601,709

(22) Filed: Nov. 20, 2006

Related U.S. Application Data
(63) Continuation-in-part of application No. PCT/IL05/00515, filed on May 19, 2005.

(51) Int. Cl. A61B 1/00

(52) U.S. Cl. 600/102

(57) ABSTRACT

The present invention provides a bolus for introducing into a ruminant animal's reticulum, comprising at least one pressure sensor (e.g. acoustic sensor and pressure transducer) configured and operable for receiving an overall pressure signal emanated by two or more signal sources in its surroundings and outputting a data stream indicative thereof; a processing utility for receiving and processing the data stream indicative of the overall pressure signal to isolate therefrom signal components from two or more defined sources and outputting one or more values indicative of a health condition of the animal; and a communication utility for receiving said one or more values and transmitting a signal corresponding thereto. The invention also provides a method for monitoring health condition of a ruminant animal and a system therefore, making use of the bolus of the invention.

This Application is a Continuation In Part of PCT International Application No. PCT/IL/2005/000515 with International Filing date of May 19, 2005 which claims priority from U.S. Provisional Patent Application No. 60/572,484 filed May 20, 2004, the content of all listed applications being hereby incorporated in their entirety.

### FIELD OF THE INVENTION

This invention is generally in the field of monitoring techniques and relates to a method and system for monitoring a condition of an animal, and a bolus used therefor.

### BACKGROUND OF THE INVENTION

Farmers of livestock, both in dairy and in beef farms, face different difficulties in trying to improve production and maintain profitability. These difficulties include, for instance, the lack of accurate information regarding the health condition of the animals. Further, dairy farmers face having to deal with a lack of efficiency due to a great waste of resources in the collective feeding process.

Several devices and methods have already been developed in an attempt to reduce the difficulties of farmers involved in healthcare of farm animals and which may allow early disease detection, continuous automated animal health supervision, constant information about animals' metabolic condition to improved feeding efficiency etc. Some such systems and methods are described in the following documents:

U.S. Pat. No. 5,984,875 describes an ingestible animal bolus for monitoring physiological parameters of animals. The bolus includes circuitry for storing a selectable identification code, for sensing a physiological parameter and for transmitting a data burst signal which includes information corresponding to the identification code and a sensed physiological parameter. The system also includes a receiver for receiving data burst signals transmitted from the bolus. A preferred parameter is temperature.

Another ingestible bolus is described in U.S. Pat. No. 6,059,733, utilized for determining a physiological state, such as a core body temperature of a ruminant animal. The bolus includes a temperature sensor and a transmitter is placed within a stomach of the ruminant animal.

U.S. Pat. Nos. 6,285,897 and 6,689,056 describe an ambulatory system for detecting, recording and analyzing physiological parameters such as pH, temperature, pressure, within the esophagus or other body lumens. The system includes an implantable sensor and radiofrequency transmitter, an external receiver and recorder and an analysis software package.

Yet, monitoring devices are utilized in human healthcare. For example, U.S. Pat. No. 6,454,720 describes a system for measuring a physiological parameter in a place within a patient's body to which a medical probe has access, the system comprising the medical probe equipped with a sensor for the parameter and means for emitting an electrical signal that represents the parameter and that is received by the sensor, to a data processing device outside the body. Examples of physiological parameters measured by the system include pressure, temperature, chemical composition, pH moisture content of a gas.

Further, U.S. Pat. No. 6,632,175 describes a swallowable data recorder medical device including a capsule comprising a sensing module for sensing a biologic condition within a body and a recording module as well as a power supply.

Yet further, U.S. Pat. No. 6,527,729 describes a method for monitoring a patient using acoustic sensors, e.g. for monitoring the progression of a disease such as heart failure, so as to warn the patient or healthcare provides of changes in the patient's condition. The method comprises the steps of sensing a physiological acoustic signal inside a patient's body at a first time period; calculating value corresponding to the energy content of a portion of the acoustic signal for the first time period; sensing the acoustic signal at a second later time period; calculating a value corresponding to the energy content of the portion of the acoustic signal for the second time period; and comparing the calculated value of the energy content of the acoustic signal for the second time period with the calculated value of the energy content of the acoustic signal for the first time period and providing an output as a function of the results of the comparison.

Further, U.S. Pat. No. 6,535,131 describe an apparatus for automatically identifying when an animal is in distress, the apparatus is adapted to receive a sound pattern produced near the selected animal and to compare it with pre-stored audio patterns corresponding to respective sounds expected to be produced by that type of animal when in various types of distress to determine the best match. When a good enough match is made, a signal is automatically sent to a remote communication unit near an attendant.

Finally, US Patent application publication No. 2003205208 describes a method and system for monitoring the physiological condition, and/or suitability of animal feed, of ruminant animals, by: sensing actions of the animal indicating a ruminating activity; and accumulating the time of the ruminating activities over a predetermined time period to provide an indication of the physiological condition of the animal, and/or of desirable changes in its feed for maximizing milk production and/or for maintaining animal health.

### SUMMARY OF THE INVENTION

There is a need in the art for quick and effective monitoring of the health condition of ruminant animals, by providing a novel bolus and a monitoring method and system using such bolus.

The present invention solves the above problem by providing a novel bolus configured and operable to process an overall acoustic signal emanated from two or more different signal sources within the animal, and output two or more values indicative of respective physiological parameters of the animal indicative of its health condition, such as heartbeat rate, respiration rate, rumination activity, etc. Heartbeat rate and respiration rate are preferred conditions to be determined in accordance with the invention.

As used herein the term “bolus” denotes any device configured to be introduced into a ruminant animal's reticulum. The bolus may be introduced into the reticulum orally, by swallowing or by manual insertion via the esophagus; or by surgical means. Other features of the bolus are detailed hereinafter.

Further, it should be understood that the term “signal sources” denotes not only sources of meaningful signals, meaningful being in the sense of deterring physiological parameters of the animal, e.g. the heartbeat, respiration, and rumination; but also sounds emanated from the surrounding which may be considered as noise. Thus, the “two or more different signal sources within the animal” may include on the one hand, one meaningful signal source, such as the rumination activity, and on the other hand noise resulting from, e.g. the movement of the medium within the cavity or the movement of the cavity within the body. The “two or more different signal sources within the animal” may also include two or more true signal sources. The present invention provides a bolus according to the invention for introducing into a ruminant animal's reticulum, comprising:
at least one pressure sensor configured and operable for receiving an overall pressure signal emanated by two or more signal sources in its surroundings and outputting a data stream indicative thereof;
a processing utility for receiving and processing the data stream indicative of the overall pressure signal to isolate therefrom signal components from two or more defined sources and outputting one or more values indicative of a health condition of the animal; and
a communication utility for receiving said one or more values and transmitting a signal corresponding thereto.

In accordance with one embodiment, the pressure sensor is an acoustic sensor.

In accordance with another embodiment, the pressure sensor is a piezoelectric sensor.

In accordance with yet another embodiment, the pressure sensor is a pressure transducer.

Further, in accordance with one embodiment, the processing utility for receiving and processing the data stream employs a physical model and algorithm adapted to process said data stream so as to isolate therefrom signal components from two or more defined sources and outputting one or more values indicative of a health condition of the animal, the processing utility being a constructional part of said bolus

The present invention also provides a method for monitoring health condition of a ruminant animal, the method comprising:
introducing into the animal's reticulum the bolus of the invention;
operating the bolus to receive and process the overall acoustic signal to thereby enable determination of the health condition of the ruminant animal.

Finally, the invention provides a system for monitoring health condition of a ruminant animal, the system comprising:
one or more boluses according to the invention, each for introducing to the reticulum of a ruminant animal;
a control system connectable to said one or more boluses for receiving said values and generating data indicative thereof to thereby enable monitoring of the health condition of said one or more animals and enable operation of said one or more boluses.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1A-1C illustrate a monitoring system according to the present invention, including an overall illustration of the system (FIG. 1A); a communication system (FIG. 1B) and the monitoring and control unit (FIG. 1C-1D).

FIG. 2 is an illustration of a bolus location in the ruminant animal's reticulum;

FIGS. 3A-3C are illustrations of an Inside Processing Unit (IPU) according to one embodiment of the invention, including a bolus and its constructional components (FIG. 3A); a block diagram of the construction of the processing unit (FIG. 3B); and a flow diagram illustrating the operation of an IPU (FIG. 3C);

FIG. 4 is an illustration of the reticulum and its division into areas according to quality of recorded acoustic signal components.

FIG. 5 is an illustration of a model underlying the signal processing and analyzing algorithm employed in accordance with the invention.

FIG. 6 illustrates two possible configurations of de-coupling techniques.

FIG. 7A-7E provide a flowchart of an exemplary Internal Processing Unit (IPU) Algorithm (in time domain) (FIG. 7A); and heartbeat analysis obtained by the algorithm of FIG. 7A, including a presentation of the original signal (FIG. 7B), of the averaged signal (FIG. 7C); of the threshold signal (FIG. 7D) and the normalized signal (FIG. 7E).

FIG. 8A-8C is an illustration of a Hilbert transform analysis of heart rate according to the present invention, including the original signal (FIG. 8A); the Hilbert magnitude (FIG. 8B) and the Hilbert imaginary part (FIG. 8C);

FIG. 9A-9C provide a flowchart of an exemplary Internal Processing Unit (IPU) Algorithm according to a more preferred embodiment of the present invention (FIG. 9A); a frequency response of a filter utilized in the IPU presented in FIG. 9A (FIG. 9B); and results of such an algorithm showing the peaks of power windowing (Curve C₁), as compared to an ECG reference signal (Curve C₂) (FIG. 9C).

### DETAILED DESCRIPTION OF EXEMPLARY EMBODIMENTS

The present invention provides a system and method for monitoring the physiological condition of ruminant animals, particularly cows, and also a novel bolus for use in such system. The invention provides an efficient and reliable solution for monitoring different physiological parameters, such as heartbeat rate, rate and depth of respiration and rumination activity, of individual animals in a herd or in a group of animals. The physiological data concerning heartbeat rate, respiration and rumination is obtained by in vivo recording and processing of measured data obtained by the use of suitable pressure sensors.

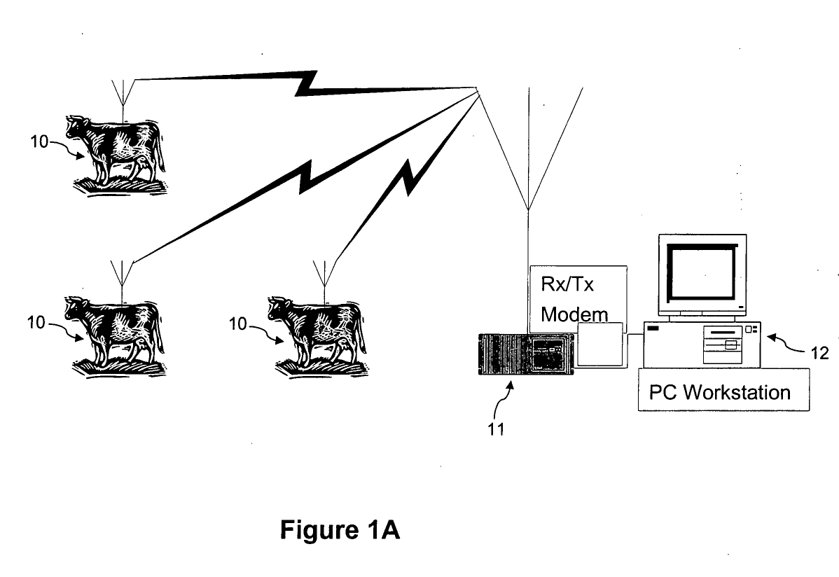

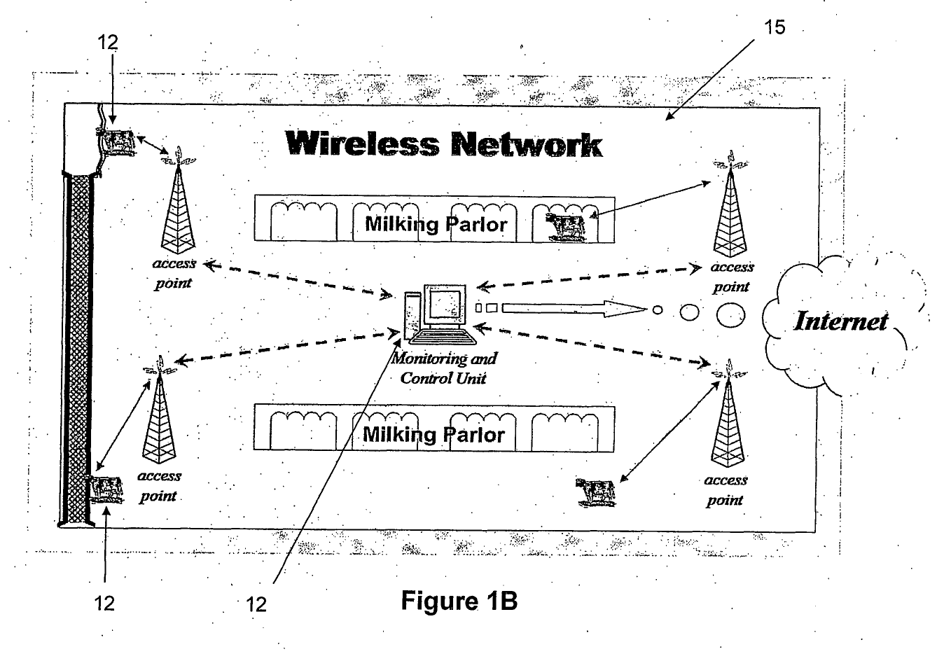

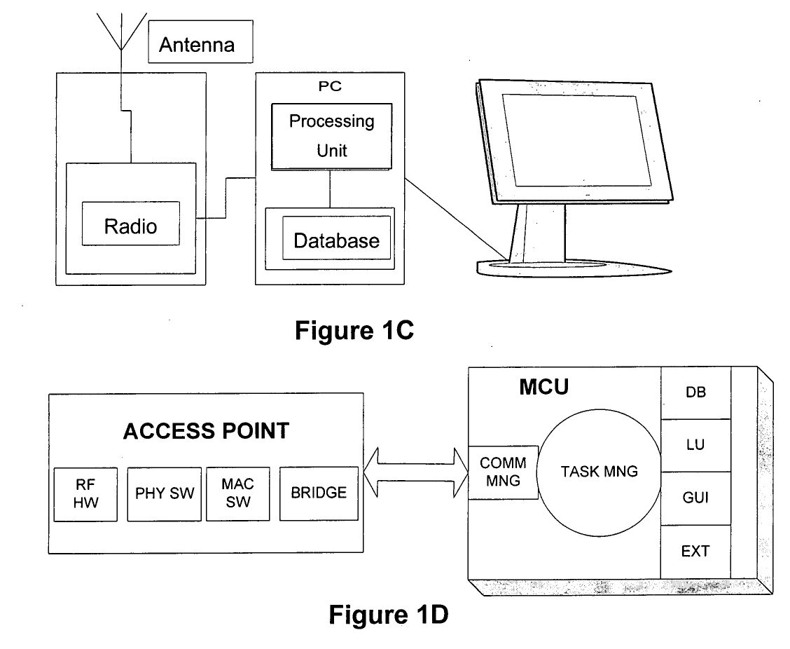

Referring to FIG. 1A, there is exemplified a monitoring system, generally at 1, according to the invention. In the present example, the system is configured for automatically monitoring a plurality of cows, generally at 10. The system 1 includes such main constructional parts as an ingestible bolus (now shown here) presenting an Internal Processing Unit (IPU) for introducing to the reticulum of one or more ruminant animals; and a monitoring utility 14 for monitoring signals transmitted from the bolus. These signals present measured data and are preferably in the form of RF transmittable signals. The monitoring utility 14 includes a control system 11 connectable to the IPU and may or may not be further connectable (via wires or wireless) to a central monitoring unit (MCU) 12.

Thus, each monitored animal 10 is equipped with the IPU that transmits measured data (which presents processed results or partially processed results of recorded data) to the MCU 12 via the control system 11. Every IPU is assigned with a unique ID that is transmitted together with an outgoing message indicative of the measured data in order to distinguish between measured data of different animals. It should be noted that the IPU communicates with the MCU 12 only and no communication occurs between different IPUs. The communication can be initiated either by the MCU 12 or by the IPU. The construction and operation of the IPU will be described further below.

The IPU is configured and operable to measure, evaluate and record the different physiological parameters of the animal such as the heartbeat rate, respiratory rate and depth, rumination duration and intervals and body temperature. The IPU may be inserted into the animal's reticulum orally or placed in its body in a surgical operation.

The communications between the IPU and MCU 12 can be performed using any known suitable technique. To this end, IPU is equipped with a suitable transmitter and MCU 12 is equipped with a suitable receiver. The system may be configured for operation of IPU from MCU, in which case IPU and MCU are equipped with both transmitter and receiver.

FIG. 1B schematically illustrates a communication system 15 (e.g., Network) that may be utilized in the present invention. Communication system 15 is formed by communication utilities (not shown) of the IPUs (located in the animals' bodies 10) and a communication utility of the MCU 12 connectable to each other via a communication network (e.g., the Internet). The main function of MCU 12 is to collect data from all the monitored animals (i.e., from all the IPUs), store the collected data, and possibly further process the data, and display the data and/or further processed results, on a simple, efficient and effective interface on the panel of the end-user.

FIG. 1C shows by way of a block diagram, the monitoring and control unit 12. Unit 12 is a typical computer system equipped with a communication utility to be connectable to a communication network. Unit 12 includes inter alia a transceiver unit 12A which enables the connection with each individual IPU (i.e., with animal), a memory utility (database) 12B, a data processing and analyzing unit 12C running a dedicated software application that performs the analysis and storage of incoming data, and an interface monitor 12D that presents the end-user all relevant data.

The software application controls and manages the radio unit 12A and all the communications with the individual IPUs, it keeps database 12B containing the information about each and every animal and the data received from all IPUs. The application also processes the information received from each IPU, analyzes it and makes a preliminary diagnosis of the animal's health, metabolic condition and estrus. This information is presented to the user on screen 12D, allowing the user to view the history, the statistics, the different data and the condition of each animal. The user may also control the system parameters such as time of data collection, the ID of the IPUs to be monitored, the number of monitored IPUs and so on. The system may also be programmed to alert the user of outstanding events such as calving alerts or of any abnormal condition of the monitored animals.

Reference is made to FIG. 1D illustrating in a self-explanatory manner a general construction of an MCU associated with one or more suitable Access Point (AP) (at this specific example one AP is illustrated although the connection to several AP's is also applicable as well). According to this particular example, the MCU includes and is controlled by a Task Manager (TASK MNG) that manages and schedules all the system's processes. It also ensures secure, robust and efficient connectivity between the following modules, also shown in the figure:

COMM—A communication module linking between the AP and the MCU.

DB—A database for recording all the animals' data and relevant information.

LU—A logic unit for processing information received and running biological algorithms in order to create applicative data for the user.

GUI—A Graphics User Interface (e.g. monitor) for accepting input from the user and displaying information using, graphs, lists, tables, etc.

EXT—An external interface module for managing the connectivity to other farm systems and the Internet.

According to this specific example, the AP comprises the following components:

RF HW—a module including the physical antenna and the hardware electronic components that convert the IPU radio signals from RF format to a baseband one.

PHY SW—a physical layer of the communications protocol.

MAC SW—a Medium Access Control of the communications protocol.

Bridge—an overall function of translating to wireless data to a wired communications format that is sent to the MCU for translation at a higher communications layer.

According to one embodiment, the AP is based on a kit model CC1010DK (commercially available from Chipcon AS, Oslo, NORWAY) consisting of an evaluation board CC1010EB coupled with an evaluation module CC1010EM. The CC1010EB is the motherboard that hosts the evaluation module with the CC1010 chip. The CC1010 integrates a very low-power 433 MHz RF transceiver and a 8051-compatible microcontroller equipped with two programmable serial UARTs (port 0 and 1). The AP connects to the MCU through a RS232 serial port connector.

The wireless communications preferably uses the 433 MHz spectrum using binary FSK modulation and Manchester encoding. The frequency corresponding to the digital “0” is denoted f₀, while f₁ corresponds to a digital “1”. The frequency separation is f₁-f₀. The RF carrier frequency, f_{c}, is then given by f_{c}=(f₀+f₁)/2. (The frequency deviation is given by f_{d}=±(f₁−f₀)/2). The frequency separation is programmable in 250 Hz steps.

In binary modulation each baud is represented by one bit per second. The communications will able to use different data rate as shown in Table 1:

TABLE 1
Wireless Communications Data Rates

| | Data Rate (bytes/sec) |
|---|---|
| Data Rate (kbaud) | with Manchester encoding |
| 2.4 | 150 |
| 4.8 | 300 |
| 9.6 | 600 |
| 19.2 | 1200 |
| 38.4 | 2400 |
| 76.8 | 4800 |

The PHY layer is implemented in the Chipcon's micro-contoller. The PHY is in charge of demodulating the received sampled baseband FSK signal to bits in RX, and modulating the binary data into FSK signal in TX.

The MAC layer is implemented in the Chipcon's micro-contoller. Each message transmitted is encapsulated in a frame of the following format:

| | | | | |
|---|---|---|---|---|
| Preamble signal | 8 bits sync word | 8 bits | Data | 16 bits CRC |
| | | Data Length | | |

The preamble signal is composed of alternating “0”and “1”using a user definable length (3, 5 or 7 bytes).

The MAC Layer controls the scheduling of frames and the RX and TX synchronization.

The MCU Communications Manager (COMM) consists of two sub-modules: Hardware driver and Data layer manager. The hardware driver must be able to communicate (read/write) with the AP through the PC's serial port. The data layer manager (DLM) is the module that controls the driver and manages the communications with the AP and the IPUs. This module's services are as follows:

1. Manage a communications session between a certain IPU and the MCU. A session is defined as the time while the IPU is associated with the MCU. Usually an IPU is disassociated, meaning that it is not in the MCU's range and it cannot upload data. Once, an IPU enters the MCU's range and it has data to upload, it associates with the MCU and only then it can transfer the information. Once, all the data has been received and verified by the DLM, the IPU gets disassociated.

2. Initiate a session upon user demand or as scheduled.

3. Identify the IPU and send the relevant data to the task manager for storage in the database.

4. Arbitrate the communications when several IPUs are associated.

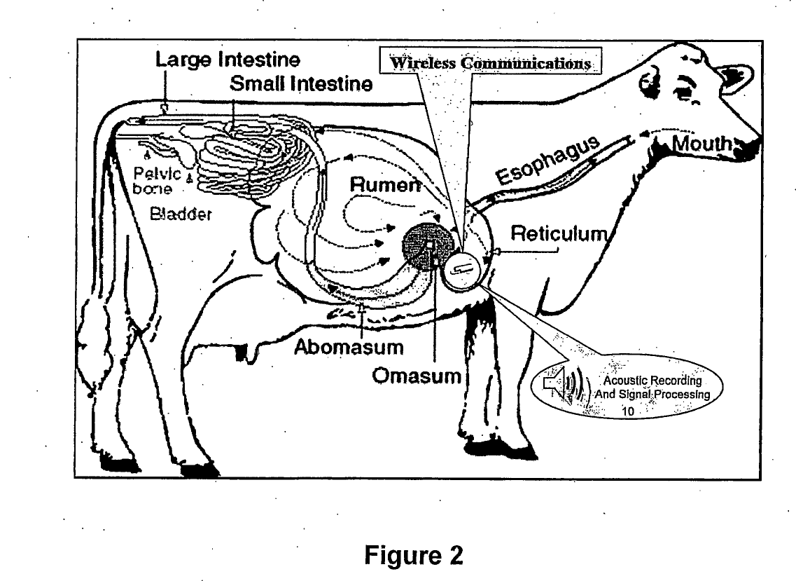

The location of a bolus (IPU) 16 in the animal's body, e.g., a cow, is illustrated in FIG. 2. The bolus 16 is preferably constructed from any known suitable biologically inert material to protect it from being eroded in the acidic environment of the reticulum (PH≈6.4). The bolus's size is small enough not to disturb or damage the animal's reticulum and large enough to prevent it from exiting the first cavity, i.e. the reticulum into the following cavity. According to one embodiment, the bolus's size is about 10 cm long and 2.5 cm wide.

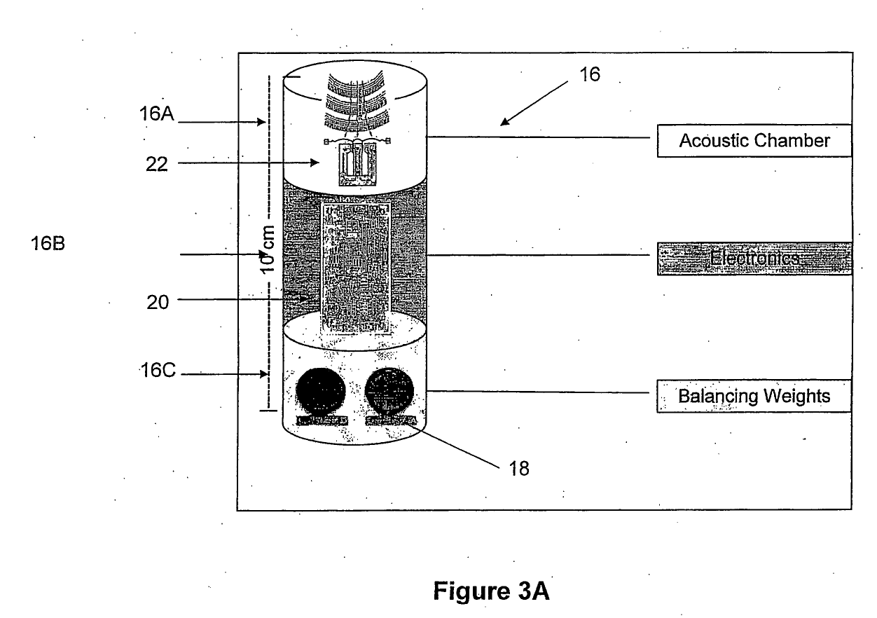

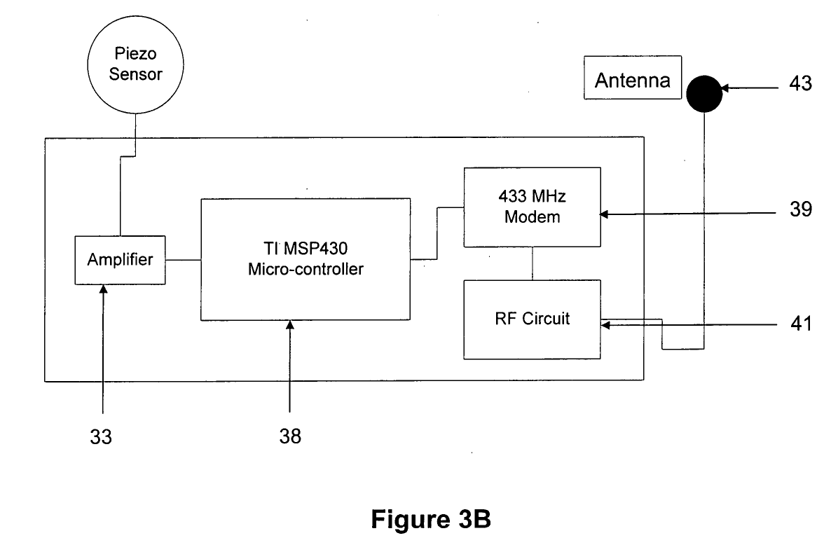

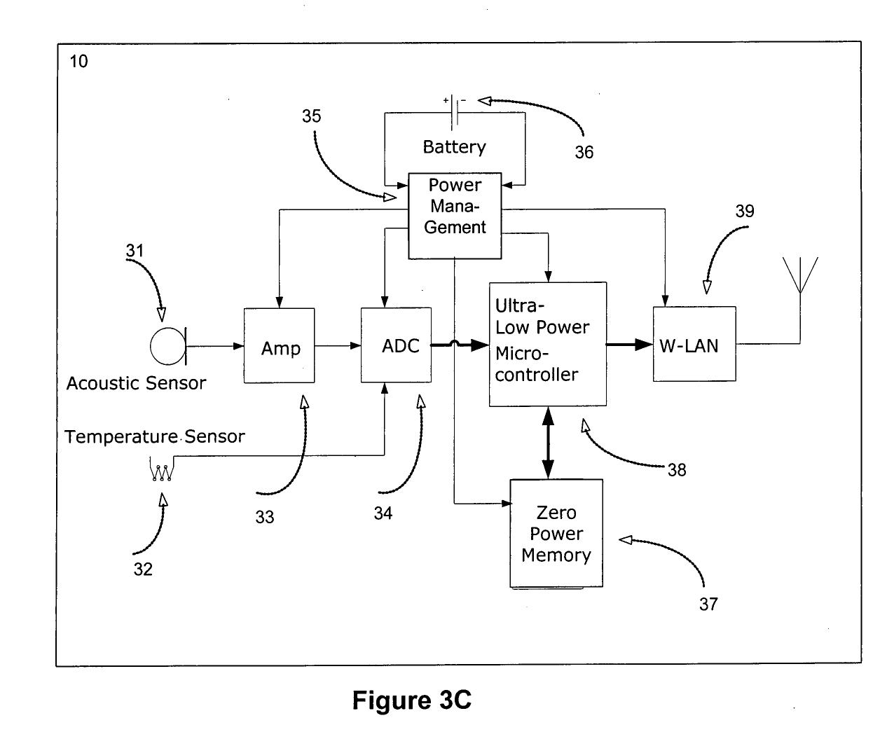

Reference is made to FIGS. 3A to 3C, there is exemplified a construction and operation bolus 16 of the present invention.

As shown in FIG. 3A, the bolus 16 is configured as a cylinder. Preferably, the cylinder has rounded edges. In the present example, the bolus has a length of 10 cm and a diameter of 2.5 cm. The bolus includes three modular compartments: a bottom compartment 16A including a ballasting assembly 18 (e.g., in the form of balancing weights), an intermediate compartment 16B including a processing unit 20, and a top compartment 16C configured as a pressure chamber 22 including one or more pressure sensors.

In the context of the present invention a pressure sensor denotes any sensor (or transducer) which measures pressure. A typical pressure sensor is that using diaphragm technology where a difference in pressure of two sides of the diaphragm is measured. A pressure transducer is understood to include any transducer that converts pressure into an analog electrical signal.

One pressure sensor in accordance with the invention is that using piezoelectric technology where a pressure causes a geometrical change of the sensor resulting potential difference.

The pressure sensors/transducers employed in accordance with the invention are preferably those with low frequency response characteristics, e.g. with a Resonant frequency at ≧100 Hz and low frequency response at 0.5 Hz.

Non-limiting examples of pressure sensors having the above characteristics include High Sensitivity Pressure Sensors/Pressure Transducer for monitoring low-level pressure pulsations such as PCB Piezotronics sensor model 106B and model 103B.

In accordance with one embodiment, the pressure sensor is an acoustic sensor. An acoustic sensor is defined as a sensor that measures acoustic (sound) waves. One embodiment of the invention makes use of an acoustic sensor which makes use of piezoelectric material (piezoelectric sensor) with High Sensitivity for pressure pulsations

It is to be noted that while the following description provides specific, non-limiting examples refer to acoustic sensor(s), it is to be understood that other types of pressure sensors are equally applicable in the context of the invention, mutatis mutandis.

According to one embodiment, a pressure sensor, e.g. an acoustic sensor is preferably located about 10-15 cm above the bottom of the reticulum and should be in an upright position. This may be implemented by the provision of ballasting assembly 18, which is in the form of balancing weights in this specific example. The balancing weights are preferably made of a magnetic material in order to attract and immobilize unwanted metal objects swallowed by the animal. It should be noted that this positioning of the bolus was determined by performing several field experiments in which an acoustic sensor was manually placed at different, predefined areas within the reticulum and the effect of its location and tilt within the reticulum on the quality of the recorded acoustic signal components was determined. Measurements were performed in a time frame of several hours per day, for a period of up to several months.

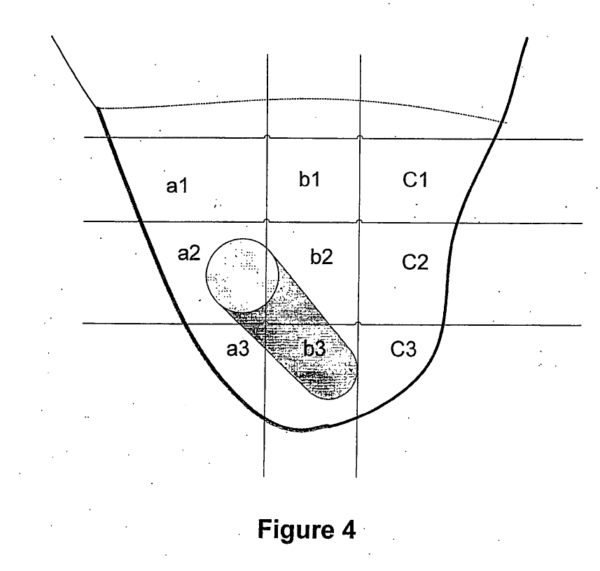

FIG. 4 is a schematic illustration of the reticulum divided into areas according to the quality of the recorded acoustic signal components. According to this illustration, reference letter “a” designates areas in which acoustic sounds emanated from the heart or the lungs were recorded at high quality; reference letter “b” designates areas in which the acoustic sounds emanated from the heart and lungs were reordered at medium quality while reference letter “c” designates areas in which the acoustic sounds were recorded at low quality,. Further, the index “1” designates areas in which the acoustic sound emanated from the lung was recorded at high quality, index “2” designates areas in which the acoustic sound emanated from the heart was recorded at high quality and index “3” designates areas in which acoustic sound emanated from the lungs was recorded at medium quality and from the heart at low quality.

It should be noted that since the bolus is freely located in the reticulum, it is subject to movement within the reticulum, e.g. due to the fluidity of the medium in which it resides, cavity movement (as a result of movement of the animal), digestion, as well as other factors. As a result, the bolus moves between the different areas within the reticulum. Thus, although the bolus may be found in areas “c” which are considered to provide signals of lower quality, normal contractions of the reticulum move the bolus into areas “a” and “b”.

The processing unit 20 includes an actual electronic board, a microcontroller (processor), an acoustic amplifier, a communication utility, and a power source. The communication utility may include a wireless communications antenna implanted in the surrounding wall of this compartment 16B.

The acoustic chamber 22 surrounds one or more acoustic sensor. The shape of this chamber is preferably rounded and made of a thin and rigid polymeric material.

The three compartments 16A-16C are appropriately attached to each other and sealed from the environmental liquids and moisture. This can be implemented using a screwing filament and an O-ring sealing.

According to a preferred embodiment, the acoustic sensor is a piezo-electric sensor. For optimal acoustic performance, the sensor is housed in a thin case made of a rigid polymer and filled with a silicone gel. Thus, the sensor itself is located inside the gel.

FIG. 3B shows by way of a block diagram the construction of the processing unit 20 in the middle compartment 16B of the bolus. The processing unit 20 includes the acoustic amplifier 33, the microcontroller 38 (e.g., MSP430 commercially available from Texas Instruments Incorporated, Dallas, Tex. USA, and the communication utility 28. The latter is formed by a wireless communications modem 39, an RF circuit 41 and antenna 43. The communication utility 28 is configured and operable for transmitting an output signal indicative of the processed results, and may also be configured to be responsive to an input interrogating signal coming from the control system to control the bolus operation.

The processing utility 20 is configured and operable for receiving and processing a data stream (overall acoustic signal) coming from the acoustic sensor to isolate signal components from two or more defined sources of the overall acoustic signal and outputting one or more values. Each of these values is based on analysis of data corresponding to acoustic signal components from at least two of the sources and is indicative of a health condition of the animal. Examples of such two or more defined acoustic sources include but are not limited to the heart, the respiratory system and the digestive system of the animal.

FIG. 3C illustrates more specifically an example of the operation of the bolus 16. The overall acoustic signal emanating from the animal's reticulum is received by the acoustic sensor 31. Also, optionally, a temperature of the reticulum media is measured by a temperature sensor 32. This overall acoustic signal is then passed through the amplifier 33 and an analog to digital converter 34 to the micro-controller unit 38 of the processing unit 20. This acoustic signal (sound) is recorded and stored in a memory utility 37 of the processing unit 20. The acoustic signal is processed in order to extract precise values of the heartbeat and the respiratory rates as well as about the rumen activity. The bolus 10 is powered by a low power energy source 36 associated with a power management utility 35. The bolus communicates with the control system (11 in FIG. 1) via the wireless communications modem 39.

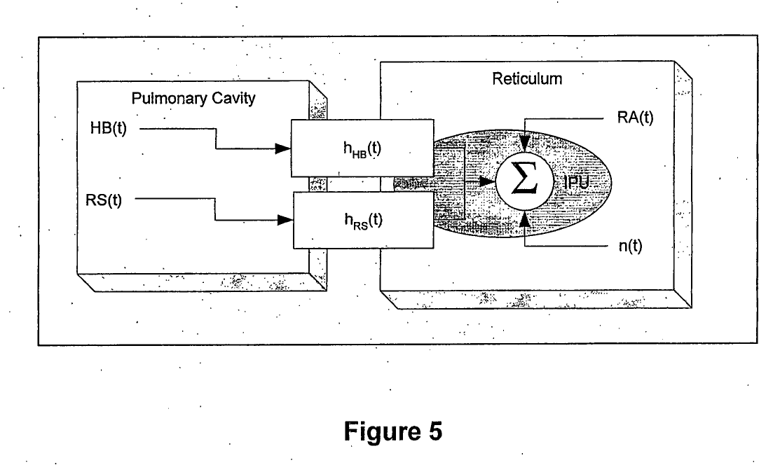

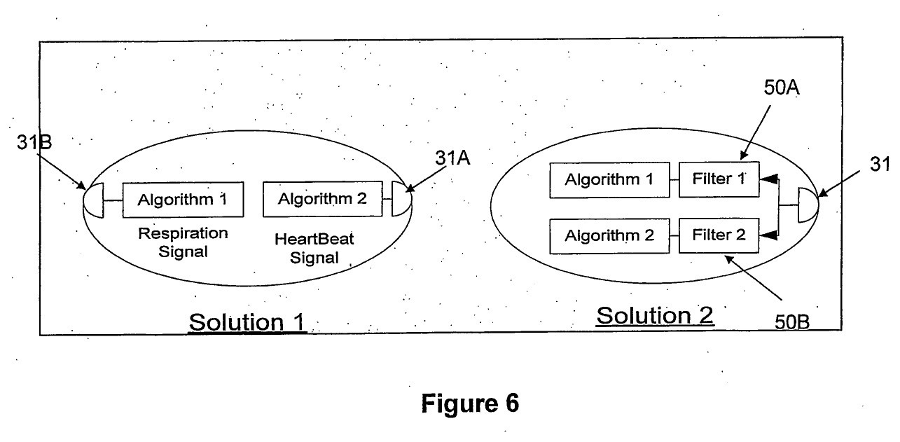

A physical model underlying the signal processing and analyzing algorithm will now be described with reference to FIG. 5. The sources of sound noticed in the reticulum include rumen and reticulum activity, heartbeat sounds and respiratory sounds from the pulmonary cavity. The in vivo auscultation of the sounds emanated in the reticulum of a ruminant animal is also characterized by a high degree of white noise. This model can be identified by the following equation:

$$s ( t ) = {\int }_{0}^{T} HB ( τ ) {h}_{HB} ( t - τ ) ⅆ τ + {\int }_{0}^{T} RS ( τ ) {h}_{RS} ( t - τ ) ⅆ τ + RA ( t ) + n ( t )$$

whereas s(t) is the overall recorded signal; HB(t) is the original heartbeat sounds; h_{HB}(t) is the impulse response of the medium through which the sounds from the heart arrive to the IPU; RS(t) is the original respiratory sounds of the lungs; h_{RS}(t) is the impulse response of the medium through which the sounds from the lungs (respiratory sounds) arrive to the IPU; RA(t) is rumen activity; n(t) is additive white Gaussian noise (AWGN).

In order to understand the components of Eq. 1, the following should be noted. The heartbeat sounds are distorted by a medium impulse response function h_{HB}(t). This distortion is manifested by the convolution between the original heartbeat sounds and the impulse response of the path the sound passes. The integral of the convolution is performed over time period T, which represents the period of observation.

The heartbeat is a non-stationary signal, meaning that the mean, the standard deviation, and all higher moments, as well as the correlation functions vary over time. However, it can be assumed that the heartbeat rate does not significantly change over short periods of time (less than one minute). It is therefore safe to consider the heartbeat as quasi-stationary. The setting of the duration of time of observation T must therefore take it into consideration, and time T should be short enough to allow the heartbeat to be considered as quasi-stationary and long enough to permit an accurate and precise calculation of the heart rate (HR). For example, the period of observation, T, should be longer than 8 seconds, but shorter than 15 seconds for optimal performance.

The medium impulse response function h_{HB}(t) is also a non-stationary function affected by the thoracic and pulmonary cavity impedance, the wall of the reticulum, the ingested matter in the reticulum, the location of the IPU in the reticulum and the acoustic sensor. The first two parameters are anatomic in essence and may be estimated in laboratory. The ingested matter is the actual food that the animal is consuming and it cannot be determined a priory; however, it is most likely that it has minor diversity.

The movement of the IPU is chaotic in the physical boundaries of the reticulum. The precise location of the IPU cannot be determined. Preferably, however, the shape and structure of the bolus are designed to assure that the acoustic sensor is always pointing upward, such as by using adequate ballasting means.

The second convolution of Eq. 1 relates to the effect of the medium on the respiratory sounds emanating from lungs of the animal. Its analysis is similar in nature to the analysis of heartbeat distortion. The non-stationary effect limitation applies as well. It must be noticed here that it cannot be assumed that h_{HB}(t)=h_{RS}(t).

While ruminating, the animal elevates the ingested food from the rumen and reticulum substrates up the esophagus to the oral cavity for mastication. The well masticated substrates are then delivered back through the esophagus to the rumen and reticulum on a regular schedule (circa 45 seconds), and fermentation products are either absorbed in the rumen itself or flow out for further digestion and absorption downstream into the omasum. This process is characterized by high levels of noise emanating in the reticulum. When this noise occurs, the signal to noise ratio (SNR) of the recorded sound is very low and no parameters extraction can be performed. The component of the rumination activity is represented in Eq. 1 by RA(t).

The system is also characterized by a high degree of additive white Gaussian noise. Through adequate filtering this noise can be removed. Some of the sources of noise in s(t) include the fermentation of matter in the reticulum, movement of the animal, movement of the bolus in the reticulum.

Solutions for the distortion of h_{HB}(t) and h_{RS}(t) are based on the following. The IPU is aimed at determining the rate of the heartbeats, the rate of the respiration and depth of the respiration, and not at extracting HB(t) and RS(t). In order to achieve this goal it is not necessary to adequately determine h_{HB}(t) and h_{RS}(t). The solution is constituted of an effective de-coupling of the signal components such as heartbeat and respiration components in a manner that will facilitate a robust inter-beat interval extraction algorithm to calculate the rates of the two physiological parameters. Furthermore, an additional algorithm is necessary in order to estimate the depth of respiration.

As exemplified in FIG. 5, the de-coupling can be achieved using a filtering module having one of the following configurations:

(1) Two or more different acoustic sensors may be used, two such sensors 31A and 31B being shown in the present example. The sensors are associated with respective utilities of processing unit 20 running suitable algorithms 1 and 2, each for enhancing one desired parameter while attenuating the other. As shown in the figure, the enhanced parameters are, respectively, respiration signal and heart beat signal.

(2) A single sensor 31 can be used, and the sensor output passes through two or more different filters, two such filters 50A and 50B being shown in the present example. Each filter is configured and operated to separate a respective part of the overall acoustic output including a signal component of interest. Such a filtering may be a frequency based filtering. Then, the separated signal parts are processed by respective algorithms 1 and 2 to determine the required parameters.

As already mentioned, the rumination is characterized by intervals of relative silence and periods of high-level noise. By constantly monitoring the energy of the overall signal s(t), each interval can be differentiated and the desired rumination parameters can be determined. Furthermore, distinguishing between rumination and non-rumination time periods is also necessary to determine the silence intervals when the additional signal processing algorithms for extracting desired parameters (e.g., heart and lungs) are performed.

Some of the sources of noise in the overall acoustic signal s(t) are the fermentation of matter in the reticulum, the movement of the animal and the movement of the IPU 16 in the reticulum.

Several different techniques are known in the art for eliminating (suppressing) noise in the system output, such as time domain analysis (Averaging and Adaptive Amplitude Thresholding), frequency domain analysis (Fourier Transform and Short-time Fourier Transform), Hilbert Transform (Instantaneous Frequency Analysis) and Wavelet Analysis (Signal Decomposition and Reconstruction). The principles of these techniques are known per se and therefore need not be described in details, except to note how these techniques can be used in the present invention:

Time Domain Averaging is based on the fact that averaging is known to reduce white noise, because it is randomly distributed throughout the signal. According to basic probability theory, the intensity of a random signal averaging of n cycles is attenuated by \sqrt{n}. The algorithm intends to extract only the heart rate and not the sound beats S₁ and S₂ of the heart or any pathological murmurs generated by the opening and closing of the heart valves (one heart beat is considered to be a pair of the S₁ and S₂ sounds). Therefore, the adequate value of n can be quite large in order to produce efficiency without affecting the possibility to identify the deterministic mechanical behavior of the heartbeats.

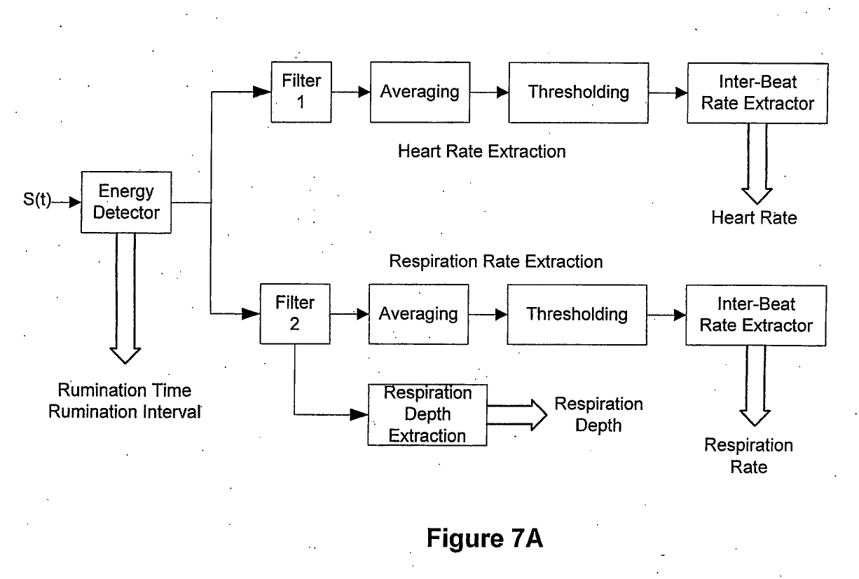

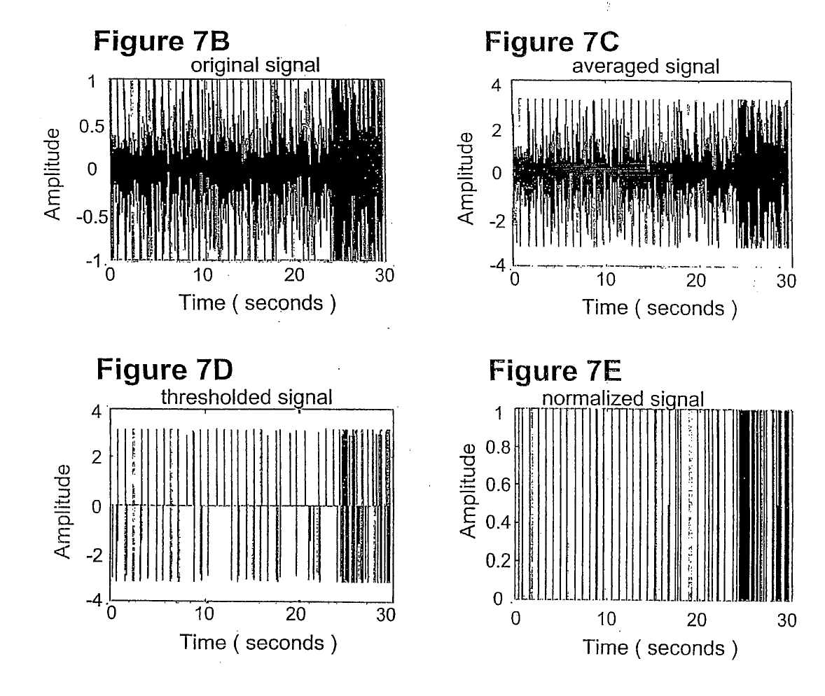

FIG. 7A is a flowchart of the Time Domain Averaging technique. As described above and shown in this figure, the first step of the algorithm detects the energy levels and distinguishes between ruminating and non-ruminating time periods—step I. The rumination time and intervals are filtered out, while non-ruminating signal components are directed for further processing—step II. During the non-ruminating time periods, heartbeat and respiration sounds may be processed and analyzed according to the following procedure: Each of filter 1 and filter 2 distinguishes between the heartbeat and the respiration sample components: filter 1 separates the heartbeat associated component (step III), and filter 2—the respiration sample associated component (step IV). To extract the heartbeat rate, the heartbeat signal component is passed through Adaptive Time Domain Averaging, the Adaptive Amplitude Thresholding and an inter-beat rate extractor—step V. Similarly, to extract the respiration rate, the respiration signal component is passed through Adaptive Time Domain Averaging, the Adaptive Amplitude Thresholding and an inter-beat rate extractor—step IV. Another parameter that can be concurrently determined is the respiration depth. To this end, filter 2 splits the respiration sample associated component into two parts, one being processed as described above for determining the respiration rate and the other being processed to calculate the respiration depth by executing the respiration depth extractor—step VII.

As for Adaptive Amplitude Thresholding, it provides the following. During the relatively silent periods of the reticulum activity the relevant signals from the heart and lungs are more intensified than the noise. In these cases, the non-linear technique of setting all of the low-level amplitudes to zero is an efficient method of noise removal. This technique is most efficient when preceded by averaging.

The results of applying Time Domain Averaging and Amplitude Thresholding techniques on a sample of heartbeat sounds in the reticulum of a cow can be seen in FIGS. 7B-7E. The original sounds as they were recorded are shown in FIG. 7B. This signal was processed by sampling the original sounds at a frequency of 8 kHz and down-sampling by a factor of 2, and then averaging, the results being shown in FIGS. 7C-7E. In this specific example, the averaging was performed on a window size of 10 samples; the threshold was set to 0.9 of the maximum amplitude. As it is evident from the graphs, the technique was very efficient. The data in the last graph (time dependence of a normalized signal) can be easily processed using an inter-beat interval algorithm in order to extract heart rate.

The Fourier Transform (FT), while being widely used in signal processing; might be less effective in this case as being not effective when used on non-stationary signals, such as heart and respiration sounds are. This is because FT does not provide frequency content information on a time scale.

Due to the fact that FT provides frequency content, but its location in time is unknown, the Short-Time Fourier Transform (STFT) has been developed. The STFT analyzes a short small section at a time called windowing. The STFT is a compromise between the time and the frequency representation of a signal providing information on a frequency when it occurs. The trade-off is between rather imprecise time and frequency resolution, which is determined by the window size. The STFT, while being less suitable for extracting the rate of a periodic beat, can be used to analyze the spectrum of the signal during a beat and distinguish between heart beats and respiration sounds.

The Hilbert Transform (HT) is usually used when instantaneous frequency attributes of a signal are important. The mathematical definition of HT is given in Eq. 2:

$$y ( t ) = {π}^{- 1} {\int }_{- ∞}^{∞} \frac{x ( τ )}{t - τ} ⅆ τ \qquad ( 2 )$$

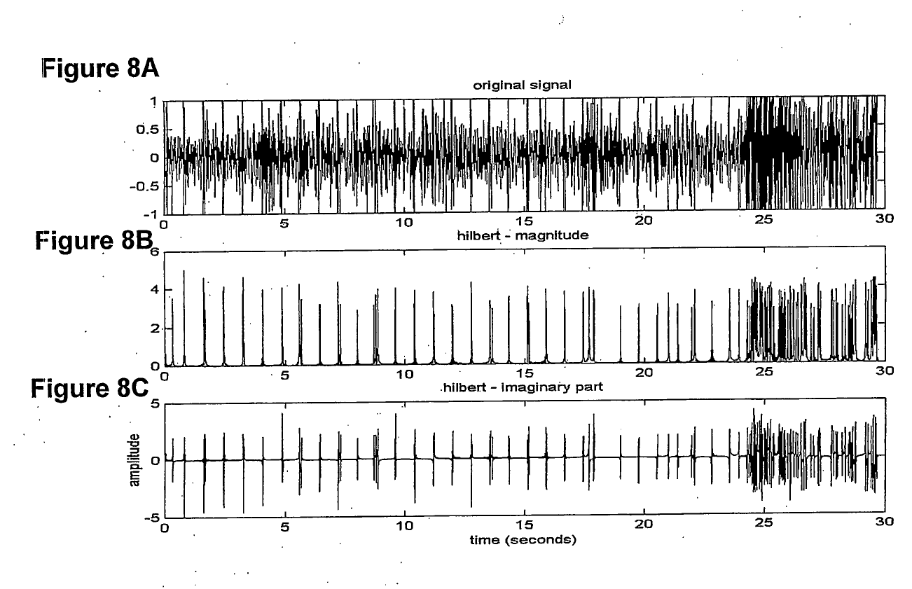

The results of HT analysis on the same data previously used in the time domain analysis are shown in FIGS. 8A-8C. As is evident from these graphs, the analysis provides a clear HT pattern, and the data can be easily processed using an inter-beat interval algorithm in order to extract HR.

The Wavelet Transform (WT) is a method for obtaining simultaneous, high resolution and frequency information about a signal. There are many factors that must be considered when using wavelets to denoise the signal in order to extract the sounds produced for example by the heart and lungs: the wavelet kernel, the size of the sample segments, the level of decomposition and the thresholding methods.

The mathematical description of the Discrete Wavelet Transform (DWT) is given in Eq. 3:

$$W ( j , k ) = {\sum }_{j} {\sum }_{k} x ( k ) {2}^{- j / 2} Ψ ( {2}^{- j} n - k ) \qquad ( 3 )$$

whereas, Ψ(t). is a time function with finite energy and fast decay termed the kernel or mother wavelet.

There are many different wavelet families presented in the literature, such as Morlet, Meyer, Daubechies, Symlets, Coiflets and so forth. The WT is suitable for denoising and analysis in the present invention, e.g., for extracting heartbeat and respiration sounds.

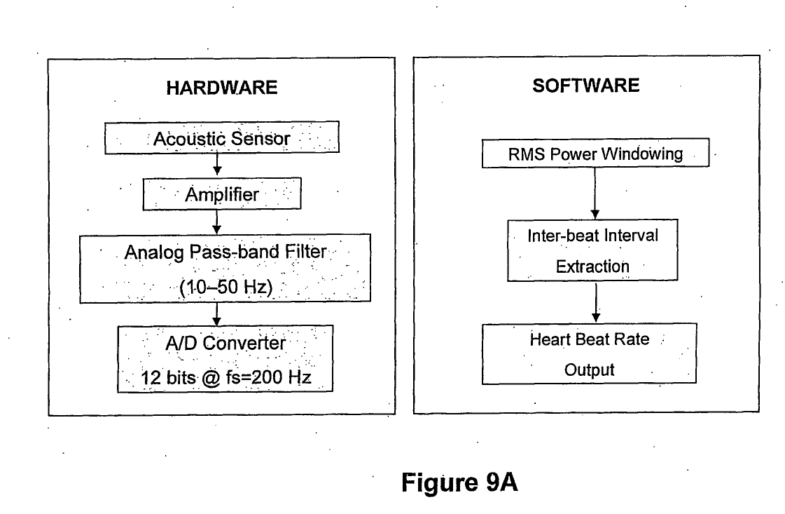

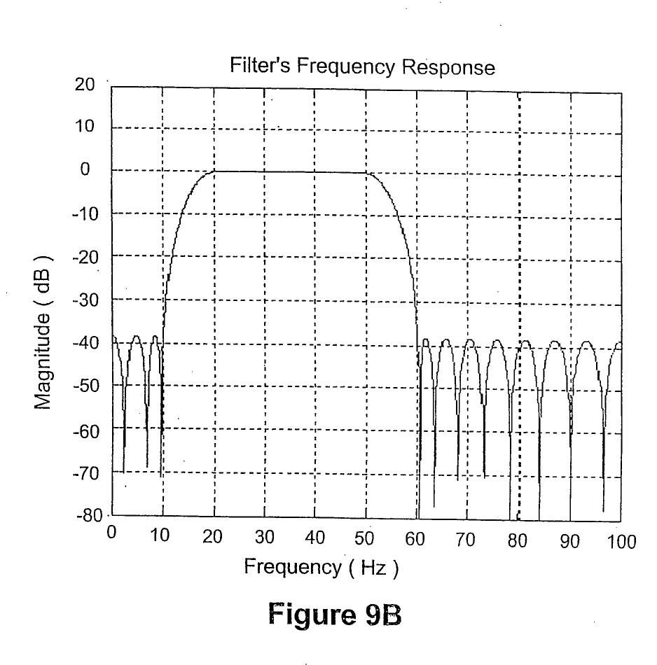

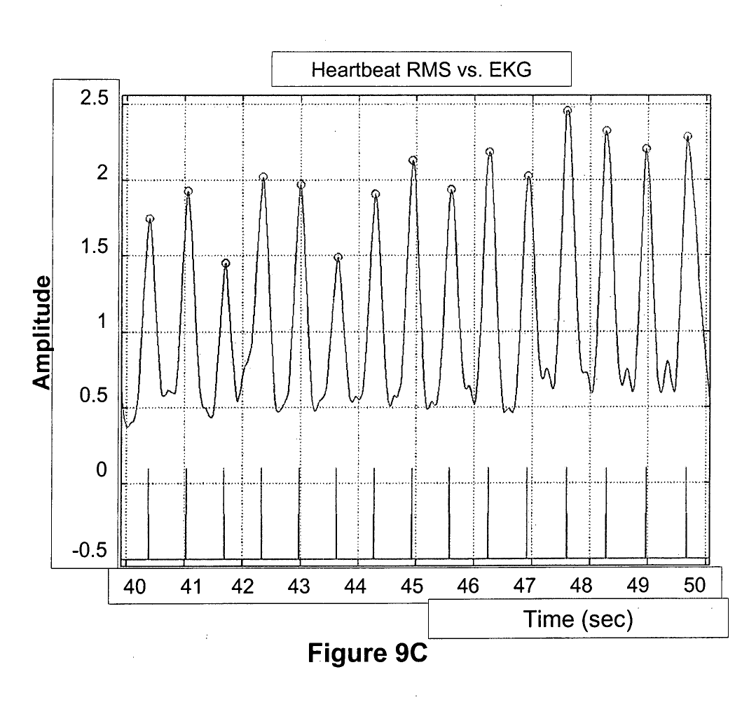

Referring to FIGS. 9A to 9C, there is illustrated more specifically a preferred embodiment of the invention utilizing a refined time-domain processing denosing algorithm. For the purpose of extracting heart beat rate. According to this particular example, a combination of software and hardware is used as shown in FIG. 9A. The hardware utility is formed by the acoustic sensor, amplifier at the output of the acoustic sensor, the analog pass-band filter (e.g., operable in 10-50 Hz) that processes the amplified output of the acoustic sensor, and A/D converter (e.g., 12 bits @ fₛ=200 Hz). The software utility is configured for processing the output digital signal of the hardware utility by applying thereto a root-mean-squared (RMS) power windowing, and processing the results by extracting the inter-beat interval to thereby obtain a heart beat rate output.

In this specific example, filter 1 is a 7 order band-pass filter with a low cut-off frequency of 10 Hz and a high cut-off frequency of 50 Hz. The heartbeats can be easily distinguished in the 10-50 Hz spectrum range, where the most intense portions of the heart sounds are located. The frequency response of this filter is shown in FIG. 9B.

The respiratory sounds are characterized by higher frequency components in the range of 60-100 Hz. The adequate filter 2 used is again a similar band-pass filter in the range of 60-100 Hz. Both filters 1 and 2 are optimal equiripple linear phase FIR filters, and were designed using the Parks-McClellan method.

Following the filtering of the frequency ranges of heartbeat and respiratory sounds, an additional smoothing of the signals is preferably carried out in order for the inter-beat rate extraction to be even more efficient. This smoothing is achieved by using a sliding root-mean-squared power-averaging window. Each window must have different lengths in order to mach the duration of the heartbeat sounds and the time of inhalation respectively.

The inter-beat rate extraction modules calculate the time intervals between heartbeat peaks and the time intervals between inhalations. Finally, the results are translated as intervals per minute (e.g., heartbeat rate and respiratory rate). The results of the algorithm are shown in FIG. 9C. Curve C₁ shows the peaks of power windowing, while curve C₂ is an ECG reference signal.

1. A bolus for introducing into a ruminant animal's reticulum, comprising:
at least one pressure sensor configured and operable for receiving an overall pressure signal emanated by two or more signal sources in its surroundings and outputting a data stream indicative thereof;
a processing utility for receiving and processing the data stream indicative of the overall pressure signal to isolate therefrom signal components from two or more defined sources and outputting one or more values indicative of a health condition of the animal; and
a communication utility for receiving said one or more values and transmitting a signal corresponding thereto.

2. A bolus for introducing into a ruminant animal's reticulum, comprising:
at least one pressure sensor configured and operable for receiving an overall pressure signal emanated by two or more signal sources in its surroundings and outputting a data stream indicative thereof;
a processing utility for receiving and processing the data stream by employing a physical model and algorithm adapted to process said data stream so as to isolate therefrom signal components from two or more defined sources and outputting one or more values indicative of a health condition of the animal, the processing utility being a constructional part of said bolus; and
a communication utility for receiving said one or more values and transmitting a signal corresponding thereto.

3. A bolus according to claim 1, wherein said processing utility comprises a filtering module configured and operable for receiving an input data and isolating therefrom data corresponding to pressure signal from two or more defined pressure sources emanating from one or more of the heart, the respiratory system and the digestive system of the animal.

4. A bolus according to claim 1, comprising two or more different pressure sensors associated with respective two or more utilities of the processing unit running two or more respective algorithms, each for enhancing one signal component while attenuating all the other.

5. A bolus according to claim 3, comprising the single sensor and said filtering module comprising two or more different filters, each filter being configured and operable to isolate from the overall pressure signal a part including the respective signal component, to be processed by a respective algorithm in the processing unit to determine the required parameters.

6. A bolus according to claim 1, wherein the analyzed data stream corresponds to pressure signals recorded over a predetermined observation period of time T that is selected to be short enough to allow the heartbeat to be considered as quasi-stationary and long enough to permit an accurate and precise calculation of the heart rate.

7. A bolus according to claim 5, wherein the time T is about 8-15 seconds.

8. A bolus according to claim 1, wherein the processing unit includes a hardware utility configured and operable to receive said data stream indicative of the overall pressure signal, apply a frequency filtering thereto, and provide a digital signal representative of the filtered signal; and a software utility preprogrammed to process the digital signal by applying thereto a root-mean-squared power windowing and inter-beat interval extraction to thereby obtain the one or more values indicative of the health condition of the animal.

9. A bolus according to claim 1, wherein the output of the processing unit includes data indicative of a unique ID assigned to the animal.

10. A bolus according to claim 1, wherein said signal generated by the communication utility is a two way radio-frequency signal.

11. A bolus according to claim 1, wherein said pressure sensor is an acoustic sensor for measuring acoustic signal.

12. A bolus according to claim 2, wherein said pressure sensor is an acoustic sensor for measuring acoustic signal.

13. A method for monitoring health condition of a ruminant animal, the method comprising:
introducing into the animal's reticulum a bolus according to claim 1;
operating the bolus to receive and process the overall pressure signal to thereby enable determination of the health condition of the ruminant animal.

14. A method for monitoring health condition of a ruminant animal, the method comprising:
introducing into the animal's reticulum a bolus comprising:
at least one pressure sensor configured and operable for receiving an overall pressure signal emanated by two or more signal sources in its surroundings and outputting a data stream indicative thereof;
a processing utility for receiving and processing the data stream by employing a physical model and algorithm adapted to process said data stream so as to isolate therefrom signal components from two or more defined sources and outputting one or more values indicative of a health condition of the animal, the processing utility being a constructional part of said bolus; and
a communication utility for receiving said one or more values and transmitting a signal corresponding thereto;
operating the bolus to receive and process the overall pressure signal to thereby enable determination of the health condition of the ruminant animal.

15. A method according to claim 12, wherein said operating comprises selecting an optimal observation time T for the bolus operation, the observation T being selected so as to be short enough to allow the heartbeat to be considered as quasi-stationary and long enough to permit an accurate and precise calculation of the heart rate.

16. A method according to claim 13, wherein said operating comprises selecting an optimal observation time T for the bolus operation, the observation T being selected so as to be short enough to allow the heartbeat to be considered as quasi-stationary and long enough to permit an accurate and precise calculation of the heart rate.

17. A method according to claim 14, wherein the time T is about 8-15 seconds.

18. A method according to claim 15, wherein the time T is about 8-15 seconds.

19. A method according to claim 12, wherein said operating comprises actuating the processing unit of the bolus for receiving and processing the data stream indicative of the overall pressure signal to isolate therefrom signal components from two or more defined sources and outputting one or more values indicative of a health condition of the animal.

20. A method according to claim 13, comprising assigning a unique identification code to the bolus to be transmitted together with said one or more values.

21. A method according to claim 12, comprising assigning a unique identification code to the bolus to be transmitted together with said one or more values.

22. A method according to claim 12, comprising operating a communication between one or more bolus and an external control system.

23. A method according to claim 13, comprising operating a communication between one or more bolus and an external control system.

24. A system for monitoring health condition of a ruminant animal, the system comprising:
one or more boluses according to claim 1 each for introducing to the reticulum of a ruminant animal;
a control system connectable to said one or more boluses for receiving said values and generating data indicative thereof to thereby enable monitoring of the health condition of said one or more animals and enable operation of said one or more boluses.

25. A system for monitoring health condition of a ruminant animal, the system comprising:
one or more boluses comprising:
at least one pressure sensor configured and operable for receiving an overall pressure signal emanated by two or more signal sources in its surroundings and outputting a data stream indicative thereof;
a processing utility for receiving and processing the data stream by employing a physical model and algorithm adapted to process said data stream so as to isolate therefrom signal components from two or more defined sources and outputting one or more values indicative of a health condition of the animal, the processing utility being a constructional part of said bolus; and
a communication utility for receiving said one or more values and transmitting a signal corresponding thereto each for introducing to the reticulum of a ruminant animal;
a control system connectable to said one or more boluses for receiving said values and generating data indicative thereof to thereby enable monitoring of the health condition of said one or more animals and enable operation of said one or more boluses.

26. A system according to claim 23, wherein the control system is wireless connectable to the bolus via a two way radio-frequency transmission.

27. A system according to claim 24, wherein the control system is wireless connectable to the bolus via a two way radio-frequency transmission.

28. A system according to claim 25, configured as a communication network enabling communication between the external control system and the communication utility of one or more bolus.

29. A system according to claim 26, configured as a communication network enabling communication between the external control system and the communication utility of one or more bolus.

30. A system according to claim 25, wherein said pressure sensor is an acoustic sensor.
